(19) United States
(12) Patent Application Publication
(10) Pub. No.: US 2025/0099409 A1
(43) Pub. Date: Mar. 27, 2025

(54) TOPICAL THERAPEUTIC

(71) Applicant: Beta Biofuel Solutions, LLC, Ringoes, NJ (US)

(72) Inventor: Paula Marie L. Ward, Ringoes, NJ (US)

(73) Assignee: Beta Biofuel Solutions, LLC, Ringoes, NJ (US)

(21) Appl. No.: 18/730,153

(22) PCT Filed: Jan. 20, 2023
(86) PCT No.: PCT/US23/61002

§ 371 Date: Jul. 18, 2024

Related U.S. Application Data
(60) Provisional application No. 63/301,804, filed on Jan. 21, 2022.

(51) Int. Cl. A61K 31/19; A61P 17/02; A61P 29/00

(52) U.S. Cl. CPC A61K 31/19; A61P 17/02; A61P 29/00

(57) ABSTRACT

A topical therapeutic product formulated for pain reduction in the injured or damaged epidermal and mucosal tissues is disclosed. The topical therapeutic may include a pharmaceutically effective dosage of sodium pyruvate incorporated into various delivery mediums including a spray, soak, an emollient salve, a lotion, a gel, a roll-on, and disposable products. In at least some embodiments, the topical therapeutic may be an admix for an existing skincare product. The topical therapeutic disclosed herein may alleviate symptoms of the body's inflammatory response to injuries and side effects of various cancer treatments providing patients with improved quality of life. Methods of using the topical therapeutic product for alleviating pain in the affected areas are also disclosed.

### CROSS-REFERENCE TO RELATED APPLICATION

This application claims a priority benefit under 35 U.S.C. § 119(e) to U.S. Provisional Patent Application Ser. No. 63/301,804 filed Jan. 21, 2022, the disclosure of which is hereby incorporated by reference in its entirety

### FIELD OF THE INVENTION

The patent document relates to a topical therapeutic that can be applied for pain reduction and alleviation in mammalian epidermal and mucosal tissues caused by injury or side effects of inflammatory response providing an improved quality of life in patients.

### BACKGROUND OF THE INVENTION

Epidermal and mucosal disorders in mammals such as dermatitis, psoriasis, canker sores, cold sores, rhinitis, sore throat, and skin burns may result from factors including trauma, disease, and degenerative conditions caused by injury, age, genetics, and environmental exposure. Epidermal and mucosal injuries typically result in symptoms including skin rash, peeling, flaking, blisters, and open sores or ulcers which are often quite painful.

Additionally, injuries to the epidermal and mucosal tissues may recruit reactive oxygen species as a part of the immune response resulting in inflammation of the injured area. A common treatment for pain reduction in these types of injuries may include steroidal compounds. However, this introduces problems such as hormone level imbalance resulting in cascading issues including but not limited to, liver problems, mental/behavioral problems, sore mouth, weakened nasal cavity, stomach ulcers, thinning of the skin that bruises easily, and delayed wound healing.

Furthermore, while commonly performed medical and surgical procedures such as chemical and radiation treatments in cancer therapy may be effective and often lifesaving, they also come with painful and adverse side effects that patients will have to endure. Treatments such as radiation therapy may induce radiation burn on the outer layer of the general epidermis. Additionally, chemotherapy may produce sores and ulcers inside the mouth that make it painful and almost impossible to eat or drink. As result, patients who go under chemotherapy are often nutritionally deficient and fall into worse health conditions.

In another application, hormone therapy may be used in treating and reducing symptoms associated with breast and prostate cancers. Hormone therapy, while maybe effective in inhibiting cancer growth, is associated with side effects including but not limited to bone loss leading to a higher risk of injury; vaginal dryness, irritation, itching; and scalp flaking and itching. Furthermore, excess hormones may be excreted from the body as urine or stool that deteriorates the rectal, urethral, and vaginal areas causing severe pain when urine or stool matter is released.

While prior treatments have attempted to reduce these epidermal and mucosal injuries, there remains a need for a topical therapeutic that provides fast, immediate pain reduction and does not adversely, or antagonistically affect the current treatment regimen. Therefore, there exists a need for a therapeutic product that is easily accessible, effectively reduces and even temporarily eliminates the pain, promotes cell and tissue repair at the injury site, and improves the quality of life in patients with short-term and prolonged epidermal and mucosal pain and injuries.

### SUMMARY OF THE INVENTION

Various embodiments provide a topical therapeutic system with a multifold of advantages for alleviating pain, promoting cell and tissue repair, and improving quality of life in a patient suffering from epidermal and mucosal injuries and inflammatory responses from reactive oxygen species production in the body.

In one aspect, the present disclosure provides for a topical therapeutic product formulated for the delivery of a pharmaceutically effective amount of sodium pyruvate to the targeted areas of injury or inflammation. In various embodiments, the delivery medium may include a spray, a soak, an emollient salve, a lotion, a gel, a roll-on, or an imbued disposable. In some embodiments, the topical therapeutic imbued disposables may include a panty liner, a wipe, an eye patch, a diaper, an adhesive bandage, and a trans-epi-dermal patch. In various embodiments, the topical therapeutic spray may include a nasal, a throat, or a body spray. In at least some embodiments, the topical therapeutic may be an admix for a skincare product. An example skincare product may include sunscreen, a moisturizer, a facial cream, a facial mask, a hand and body lotion, and a scalp moisturizer.

In another aspect, the present disclosure provides a therapy method for alleviating epidermal and mucosal injuries in a patient. In various embodiments, the method may include administering the topical therapeutic to the injury area, then delivering a pharmaceutically effective amount of the topical therapeutic to the surface of the injury area to provide pain reduction. In some embodiments, the method may further include re-administering the topical therapeutic to the injury area to provide continuous pain reduction.

In another aspect, the present disclosure provides a pharmaceutically effective aqueous solution of sodium pyruvate salt. In some embodiment, an effective concentration of sodium pyruvate may be between about 100 mg/L to about 500 mg/L, about 150 mg/L to about 350 mg/L, and about 200 mg/L to about 250 mg/L. In some embodiments, sodium pyruvate solution may be dosed in saline. In other embodiments, the sodium pyruvate solution may be dosed in saline and may further include about 5 wt % to about 10 wt % of glycerin.

The details of one or more aspects of the disclosure are set forth in the description below. Other features, objects, and advantages of the technique described in this disclosure will be apparent from the description and from the claims.

### DETAILED DESCRIPTION

Various embodiments of this patent document provide a product for therapies for treating damaged mammalian epidermal and mucosal tissue cells involving inflammatory response resulting from reactive oxygen species production and tissue injuries. The inflammatory response mediator and the various therapeutic products disclosed herein reduce the undesirable cascade effects of the body's inflammatory response and provide direct pain reduction on the injured area and improve the quality of life of a patient suffering from naturally occurring conditions or undergoing various treatments and therapies.

While the following text may reference or exemplify specific formulation and delivery systems, it is not intended to limit the scope of the invention to such particular references or examples. For example, other delivery mediums for the topical therapeutic product may be contemplated. The resulting products will achieve the same objective by reducing pain and promoting cell and tissue repair in the administered area where epidermal or mucosal injury has occurred.

Various modifications may be made by those skilled in the art, in view of practical and economic considerations, such as the source and the compatibility of the materials used for the delivery medium and the characteristics of various epidermal and mucosal tissue injuries. In order to more clearly and concisely describe the subject matter of the claims, the following definitions are intended to provide guidance as to the meaning of the terms used herein.

### Definitions

The articles “a” and “an” as used herein mean “one or more” or “at least one,” unless otherwise indicated. That is, reference to any element of the present invention by the indefinite article “a” or “an” does not exclude the possibility that more than one of the elements is present.

“About” means the referenced numeric indication plus or minus 10% of that referenced numeric indication.

“Admix” refers to the incorporation of disclosed sodium pyruvate into an existing product to provide added functionality.

“Antagonistic effect” refers to an interaction between two or more drugs or treatments that have the opposite effect on the body that may block or reduce the effectiveness of one or more of the drugs or treatments.

“Antioxidant” refers to a substance that helps protect the skin's surface from oxidative damage.

“Applying,” “application,” “administering,” or “administration” refers to the application of the topical therapeutic to the affected and adjoining areas of epidermal and mucosal tissue cells by spreading, gentle rubbing, spraying, or allowing extended contact with the affected and adjoining areas.

“Aqueous solution” refers to a solution in which a solute is dissolved in water.

“Cascade effect” refers to a chain of unforeseen or unexpected events that may cause additional adverse effects.

“Colloid bath” refers to a bath prepared by adding soothing agents to bath water to relieve skin irritation.

“Delivery medium,” or “delivery system” refers to the mode of administering the topical therapeutic compound to the affected areas of epidermal and mucosal tissue cells.

“General epidermis” refers to the outermost layer of skin on the body.

“Micelle” refers to a mediator that allows an epidermal penetration of sodium pyruvate formulation in a delivery medium.

“Patient” or “subject” refers to both human and non-human mammals undergoing therapies or treatments.

“Pharmaceutically acceptable salt” refers to a salt compound that possesses the desired pharmacological or therapeutic activity.

“Pharmaceutically effective” or “effective” amount refers to an amount that is nontoxic but sufficient of sodium pyruvate to provide the desired therapeutic effect

“Reactive oxygen species” refers to a type of unstable molecule that contains oxygen and reacts with other molecules in a cell during the body's immune response to injury.

“Superoxide” refers to a type of reactive oxygen species that reacts with other molecules in a cell during the body's immune response to injury.

“Surfactant” refers to an additive to an aqueous solution to facilitate integration into an oil-based or non-polar product.

Unless otherwise specified, all percentages are percentages by weight.

Before discussing the details of the relevant disclosed embodiments, it is helpful to understand the mechanism of the inflammatory response of the body.

When the disruption of epidermal and mucosal tissues cells in mammals is induced via disease or injuries, the activated macrophages release inflammatory mediators including nitric oxide and other reactive oxygen species to induce vasodilation which widens the blood vessel resulting in increased blood volume while slowing down the blood flow. This reduced blood flow enables immune cells in blood to leave the circulation and enter the damaged tissue. Additionally, nitric oxide and other reactive oxygen species also increase vascular permeability resulting in blood fluid leaking into the infected tissues. The local concentration of fluid and plasma proteins helps concentrate proteins in infected tissue. Accordingly, this influx of fluids is associated with symptoms of inflammation such as swelling, redness, and heat in the injured area.

Further, during the inflammatory response, a large amount of superoxide may be produced. Superoxide is responsible for the vasoconstriction of blood vessels and the killing of pathogens in the body's immune response but is also associated with adverse side effects such as oxidative burst in cells and tissue damage when in excess.

Antioxidant molecules such as pyruvate-based compounds that may act as a remedy to the undesirable effects of inflammatory response from reactive oxygen species are contemplated. Sodium pyruvate, also known as pyruvic acid sodium salt, alpha ketopropionic acid sodium salt, or 2-oxopropanoic acid sodium salt, is a potential candidate due to its commercial availability, therapeutic properties, and is naturally produced in the body during glycolysis. Furthermore, the antioxidant properties of sodium pyruvate may produce favorable interactions with the injured cells and tissues to counter excessive superoxide production, thus promoting cell and tissue repair.

The acid form of pyruvate, also known as pyruvic acid, is widely used in dermabrasion and surface skin repair treatments. This iteration of treatment utilizes pyruvic acid compound in millimolar concentrations, which corresponds to the concentration in which the body uses it for de novo skin repair. The various embodiments of the present disclosure describe products formulated from sodium pyruvate in solutions ranging from about 100 mg/L to about 500 mg/L, about 150 mg/L to about 350 mg/L, and about 200 mg/L to about 250 mg/L. The time-dependent relief effects correlate to molar concentration depending on the specific topical application, and the co-ingredient design.

The following discussion includes a description of a topical therapeutic for reducing pain in the injury area, alleviating inflammatory symptoms, and promoting cell and tissue repair in accordance with the principles of the present disclosure. Alternate embodiments are also disclosed.

### Topical Therapeutic Products for Alleviating Pain in Epidermal and Mucosal Tissues

An aspect of the patent document provides a topical therapeutic product and a method of therapy for alleviating pain in the mammalian epidermal and mucosal tissues. The various topical therapeutic embodiments disclosed herein may include a pharmaceutically acceptable sodium pyruvate salt formulation that is solubilized and incorporated in various delivery mediums or products that can be topically applied to the injury area. Modes of topically administrating the disclosed topical therapeutic include but are not limited to oral and nasal inhalation via a spray, application of the topical therapeutic on the injured area of the general epidermis, and target-directed application via therapeutic imbued disposable products.

In some embodiments, sodium pyruvate salt may first be solubilized into a pre-composed product as an aqueous fraction of the product formulation to maintain the moisture percentage of the formulation, then further blended with the product. In various embodiments, topical therapeutic products may be water-based or oil-based, depending on dosage or the delivery medium. In some embodiments, sodium pyruvate salt may be dissolved in water or a pharmaceutically acceptable polar solvent for incorporation into water-based products. In another embodiment, sodium pyruvate salt may be first dissolved in water or a polar solvent, then incorporated into an emulsion, dispersion, gel, or hydrogel for integration into oil-based products. In various embodiments, the topical therapeutic product may be primarily a lipid. In this case, sodium pyruvate solution may be formulated with a surfactant to create a micelle for incorporation into a lipid product environment. In an example embodiment, about 5 wt % to about 10 wt % of glycerin may be integrated with sodium pyruvate as an emollient. In some embodiments, sodium pyruvate salt may be incorporated into the aqueous or polar phase of a topical therapeutic product having both polar and non-polar phases.

In some embodiments, a concentration of the sodium pyruvate formulation dosed in saline or saline and glycerin may be produced. This embodiment is a concentrated masterbatch sodium pyruvate formulation in which a pharmaceutically effective amount of the masterbatch may be incorporated in various delivery mediums or products for commercial production. According to one masterbatch embodiment, the sodium pyruvate may be a dry salt product to solubilize in solution to provide a topical therapeutic product in situ.

In various embodiments, disclosed topical therapeutic may be delivered epidermally. In some embodiments, the topical therapeutic is directly applied to the skin as a lotion, emollient salve, gel, or roll-on. In some embodiments, the topical therapeutic may be delivered via a mucosal route. In an example embodiment, topical therapeutic may be delivered via a nasal spray or an oral spray.

At least in some embodiments, the topical therapeutic is an imbued disposable such as a trans-epi-dermal patch, an eye patch, a disposable wipe, a diaper, an adhesive bandage, or a panty liner.

In some embodiments, the topical therapeutic is an admix for existing skin care products such as sunscreen, a moisturizer, a facial cream, a disposable facial mask, a hand and body lotion, and a scalp moisturizer.

In some embodiments, the topical therapeutic is a soak. In some embodiment, kits are provided containing a dry pyruvate salt, a solvent, and optionally, saline or glycerin, for mixing the components in situ for an immediate topical therapeutic soak to the injured area.

Although the exact dosage will be determined on a therapeutic basis, in most cases, some generalizations regarding the dosage may be made. The present disclosure describes a product formulated from sodium pyruvate in solution ranging in millimolar concentration between about 100 mg/L to about 500 mg/L, about 150 mg/L to about 350 mg/L, and preferably about 200 mg/L to about 250 mg/L.

In cases of administration of pharmaceutically acceptable salt, dosages may be calculated as the free acid. The time-dependent relief effects correlate to molar concentration depending on the specific topical application, and the co-ingredient design. In some embodiments, the disclosed topical therapeutic may be administered 1 to 4 times a day, or as needed depending on the severity and the frequency of the injury or inflammation.

In some embodiments, multiple concentrations of the same delivery medium may be provided depending on the severity of the injury or inflammation. In some embodiments, a lower-dosage topical therapeutic may be available over the counter, while a higher-dosage topical therapeutic may require a physician's approval.

### Therapies for Products

An aspect of the patent document provides a topical therapeutic product and a method of therapy for alleviating surface injuries to the epidermal and mucosal tissues. The various topical therapeutic embodiments disclosed herein may include a pharmaceutically acceptable pyruvate salt formulation that is incorporated in various administration or delivery mediums that can be topically applied to the injury area. Multiple methods of topically administrating the disclosed therapeutic include, but are not limited to an inhalation via aerosol, application of the therapeutic product on the injured area of the general epidermis, and target-directed application at least via a therapeutic imbued disposable.

The disruption of epidermal and mucosal tissues due to injuries recruits reactive oxygen species as part of the immune response. Such reactions to the body's inflammatory response can be caused by, but are not limited to, impacts; bruising; chemical, radiation, electrical, and thermal burns; diseases such as dermatitis, psoriasis, canker, and cold sores; bodily fluid exposures to surface tissues; environmental exposures such as UV radiation, and plant chemistry sensitivities. In various embodiments, dermatitis may be atopic dermatitis, contact dermatitis, or seborrheic dermatitis. In at least some embodiments, surface burns may be a first-degree burn or a second-degree burn.

These injuries may be present anywhere on the body surface, including but not limited to the general epidermis, scalp, nasal nostril cavities, mouth, throat, eyes, semen excretory site, urine excretory site, stool excretory site, and vaginal site.

An aspect of the patent document discloses pain-reducing therapies caused by these injuries and similar inflammatory responses with the invention of the present disclosure.

In various embodiments, injury to the epidermal and mucosal area may be from a side effect of various medical treatments including, but not limited to hormone therapy, monoclonal antibody therapy, chemotherapy, and radiation therapy. In some embodiments, a patient undergoing radiation cancer therapy may exhibit dermal side effects such as itchiness, sores, peel, and radiation burn. In various embodiments, the injury to the skin may be from chemical sensitivity to plant oils such as, but not limited to poison ivy, poison oak, and other toxic or allergic plants. In at least some embodiments, the injury may be a sunburn from prolonged exposure to the sun.

A patient undergoing hormone therapy is provided with a high concentration of a hormone that may be excreted as body fluids such as sweat, semen, vaginal discharge, urine, and stool. The presence of excess hormones in the excreted body fluids damages the excretory sites such as sweat glands, urethra, vagina, rectum, and anus. Additionally, hormone therapy is associated with increased dryness and inflammation in the eye area. Furthermore, the damaged excretory sites may be further inflamed causing additional pain and medical issues. The topical therapeutic provides targeted alleviation and pain reduction to these vulnerable areas.

In some embodiments, a broad coverage of the topical therapeutic in form of lotion, gel, or a body spray may be applied to the general epidermis providing pain reduction at the sweat glands. In other embodiments, the topical therapeutic imbued disposable or trans-epi-dermal patch may be applied to the targeted areas such as, but not limited to the area around the anus or vagina, and the area near the eye. In some embodiments, therapeutic imbued wipes may be used on the affected area as part of a cleanup process after a patient excretes urine or stool.

In various embodiments, a patient undergoing radiation therapy may have radiation burns on the irradiated areas of the skin as well as exhibit dryness, itchiness, peeling, swelling, and open sores. The various embodiments of the topical therapeutic may provide alleviation to these areas providing both pain reduction and protection from additional infection. In some embodiments, broad coverage of the topical therapeutic in the form of a lotion or a gel may be applied to the general epidermis providing pain reduction at the skin surface. In other embodiments, a topical therapeutic imbued disposable or trans-epi-dermal patch may be applied to the target areas. In at least some embodiments, topical therapeutic imbued wipes may be utilized as an alternative to a colloid bath.

Further, a patient undergoing cancer therapy may develop sores in the oral cavity and throat that may inhibit them from consuming food and other nutrients due to the weakened immune system. In this case, the patient may not receive the necessary nutrients to sustain them through the cancer treatment and may lose a significant amount of weight as a result. As such, the vitality of the patient may be significantly reduced, and the cancer treatment may be less effective. To prevent this undesirable cascade effect, an oral spray may be administered to alleviate pain to the injured area in the mouth and throat allowing the patient to painlessly consume the necessary food and nutrients without discomfort thus improving the quality of life for a patient undergoing cancer treatment. In another embodiment, a nasal spray may be used to reduce pain in a patient with injuries and sores in the nasal cavity, providing a clear nasal pathway for breathing.

In various embodiments, topical therapeutic may be incorporated with daily regimens such as skincare. In some embodiments, the topical therapeutic may be an admix for existing skincare products such as a sunscreen, a moisturizer, a facial cream, a disposable facial mask, a hand and body lotion, and a scalp moisturizer to provide alleviation from injuries induced by environmental exposures such as UV radiation that causes sunburn, and poisonous or allergic plants that induce an inflammatory response to the contact area.

The topical therapeutic disclosed above may be administered prior to, subsequent to, or concurrently with other treatments and therapies. The topical therapeutic disclosed above provides beneficial pain reduction, promotes cellular and tissue repair, and improves the quality of life in a patient suffering from various skin injuries and disorders. Accordingly, the topical therapeutic disclosed may be administered with drugs and therapies without added antagonistic effects.

Various embodiments of the present disclosure may be applied to non-human, mammalian subjects. In some embodiments, the subject is household pets including but not limited to dogs, cats, hamsters, and non-human primates. In various embodiments, the subject is larger livestock including but not limited to cows, horses, pigs, goats, and sheep. Common diseases including but not limited to pruritus, inflammatory bowel disease, intervertebral disc disease, and other dermal conditions from ragweed pollen and non-venomous inject bites induce an inflammatory response in the non-human mammalian cells which manifest at least as irritation, dryness, warts, lumpy skin, and general pain and discomfort in animals. Currently, the only available treatment for alleviating these symptoms is steroid-based oral and topical products which are associated with adverse side effects including but not limited to frequent urination, diarrhea, constipation, increased agitation, aggressive behavior, and medication-induced diseases. Various embodiments and methods discussed herein may be used in veterinary medicine as an alternative to steroid-based treatments.

In various embodiments, a topical therapeutic may be administered to the injured or inflamed undercarriage, ears, snout, mouth, gum, and anal and rectum areas to reduce irritation, swelling, and pain. In some embodiments, the topical therapeutic may be a spray to cover a larger affected area. In other embodiments, the topical therapeutic may be a soak or wipes that may be applied as part of bathing or a cleaning process.

### EXAMPLES

### Example 1: Patient A

Set up: The trial consisted of administering a topical therapeutic spray to a patient suffering from rhinitis, and burning in the nose and throat resulting from persistent and long-term exposure to secondhand cigarette smoke. About 250 mg/L concentration of sodium pyruvate incorporated spray was administered to the nasal cavities and throat at about 1 ml per spray, administered two to three sprays per administration.

Summary: Patient A exhibited reduced burning or itching in the affected nasal and throat areas within 15 minutes after the first administration. The spray treatment was re-applied a couple of hours later. The second treatment provided a complete alleviation of the burning to both the nose and throat. The spray was applied prophylactically before bed. The pain and burning never returned.

### Example 2: Patient B

Set up: The trial consisted of administering a topical therapeutic wipe to patients suffering from epidermal disturbances from psoriatic plaques. Patient B exhibited psoriatic plaques on his back. About 250 mg/L concentration of sodium pyruvate formulated with glycerin emollient imbued was administered to the affected area via an imbued wipe. The treatment was applied twice a day, once in the morning and once in the evening.

Summary: Patient B exhibited reduced burning or itching within 15 minutes after administration. With continuous administration, itching and burning did not return, and the skin surface returned to normal form. When the topical therapeutic treatment was interrupted, the plaques returned along with itching and burning.

### Example 3: Patient C

Set up: The trial consisted of administering a topical therapeutic spray to a patient suffering from epidermal disturbances from psoriatic plaques. Patient C exhibited psoriatic plaques behind her ears and the scalp area. About 250 mg/L concentration of sodium pyruvate formulated with glycerin emollient was administered to the affected area via an imbued roll-on. The treatment was applied twice a day, once in the morning and once in the evening.

Summary: Patient C exhibited reduced burning and itching within 15 minutes after administration. With continuous administration, itching and burning did not return and the skin surface returned to normal form. When the administration was interrupted, the plaques returned along with itching and burning.

### Example 4: Patient D

Set up: The trial consisted of administering a topical therapeutic spray and wipe to a breast cancer patient suffering from redness and burning on the general epidermis and deep muscle pain post-radiation therapy treatment. The patient received 30 daily radiation treatments in just over a month. The medical staff recommended the patient keep the skin moisturized via a petrolatum product which did not provide any relief from the pain, burning, and redness. About 250 mg/L concentration of sodium pyruvate formulated with glycerin emollient was administered to the affected areas via imbued wipe and spray

Summary: Patient D exhibited a reduction in burning without any blistering to the skin within 30 minutes after the first administration via an imbued cloth wipe. The topical therapeutic spray was used afterwards ad hoc. After topical therapeutic treatments and the follow-up visits, the radiation oncologist remarked how unusual and unexpected the usual browning of the affected area dissipated more rapidly than usual.

### Example 5: Patient E

Set up: The trial consisted of administering a topical therapeutic imbued wipe to a patient receiving a concurrent Taxotere and Carboplatin chemotherapy, with monoclonal antibody infusion treatments. The side effects of cancer treatment presented the patient with severe burning, itching, and peeling in the urethral, vaginal, and anal areas due to the elimination of the chemicals via urine and stools. About 250 mg/L concentration of sodium pyruvate formulated with glycerin emollient wipe was used to clean the affected areas after elimination, in addition to when the discomforts were present.

Summary: Patient E exhibited reduced burning and itching within one hour after the initial administration. The sloughing of damaged skin ceased within a few days of continued administration. Patient E continued using the topical treatment concurrently with cancer therapy, and for about a month after.

### Example 6: Patient F

Set up: The trial consisted of administering a topical therapeutic spray to a 5-year-old Jack Russel Terrier suffering from redness, itchy splotches on the underside of the body, and fur-less areas around the ears due to ragweed pollen sensitivity. The dog exhibited visible discomfort and scratching resulting from the injury. After cleaning with water and drying the affected areas, about 250 mg/L concentration of sodium pyruvate spray formulated with glycerin was applied to the undercarriage and ears.

Summary: After the first administration, the redness subsided, and the dog relaxed within 15 minutes. The treatment was administered after each time the dog went outside and continued throughout the ragweed pollen season. During the pollen season, when the administration was interrupted, the itching, redness, and scratching returned.

Example 7: Patient G

Set up: The trial consisted of administering a topical therapeutic spray to a cancer patient receiving Taxotere and Carboplatin chemotherapy. The side effects of cancer treatment presented the patient with severe mouth sores, pain, and skin eruptions which prevented regular mastication and intake of food and some beverages. Patient G's tastebuds were also severely affected, and the taste was unreliable. While the cancer industry experts recommended applying baking soda to the affected areas, it did little to alleviate the problem, leaving the patient for 18 to 20 weeks without relief. About 250 mg/L concentration of sodium pyruvate oral spray was administered inside the mouth and throat at 1 ml per spray, two to three sprays per administration.

Summary: Patient G exhibited almost complete relief of pain and burn within 30 minutes after the first administration. The product was used ad hoc to provide continuous comfort as saliva reintroduced the chemicals causing discomfort. With the use of the topical therapeutic spray, the sores resolved, and the pain and discomfort were managed. Patient G was able to regularly consume food and drinks without discomfort. However, issues with taste were not improved with the topical therapeutic spray.

### Example 8: Patient H

Set up: The trial consisted of administering a topical therapeutic spray to a patient with moderate sunburn on the face, shoulders, and arms early in the summer season while working on a field project. The patient had used sunscreen, but due to the prolonged exposure, the effects of the sunburn were present. The face was particularly red as it was directly exposed to ultraviolet (UV) radiation and not covered by clothing. About 250 mg/L concentration of sodium pyruvate formulated with glycerin emollient spray was applied to the affected areas.

Summary: Patient H exhibited reduced burning within 15 minutes after the first administration. The spray was applied for another day until the redness subsided. The product aided in the prevention of blistering and peeling of the skin due to sunburn, which uncharacteristically did not occur.

### Example 9: Patient I

Set up: The trial consisted of administering a topical therapeutic spray to a patient exhibiting redness, burning, and skin inflammation after exposure to irritating vegetation, e.g., poison ivy. The affected area was cleaned with cold water without soap to remove the oils and waxes transferred from the plant. About 250 mg/L concentration of sodium pyruvate formulated with glycerin emollient spray was applied to the affected areas of the general epidermis.

Summary: Patient I exhibited reduced burning and itching within 15 minutes after the first administration. The topical therapeutic spray was readministered ad hoc depending on the severity of the exposure.

### Example 10: Patient J

Set up: The trial consisted of administering a topical therapeutic spray to a patient suffering from a burning throat due to atmospheric dryness, allergic reaction, or disease state reaction. About 250 mg/L concentration of sodium pyruvate oral spray was administered inside the mouth and throat at 1 ml per spray, two to three sprays per administration.

Summary: Patient J exhibited reduced burning, itching, and general discomfort within 15 minutes after the first administration. The product was used ad hoc to provide continuous comfort.

The foregoing merely illustrates the principles of the disclosure, is not intended to be limiting, and merely sets forth some of the many possible embodiments for the appended claims. Those skilled in the art will readily recognize various modifications and changes that may be made without following the embodiments described herein, and without departing from the spirit and scope of the following claims.

What is claimed is:

1. A topical therapeutic product formulated for pain reduction in epidermal and mucosal tissues in mammals comprising a pharmaceutically effective dosage of sodium pyruvate incorporated in a delivery medium.

2. The topical therapeutic product of claim 1, wherein the delivery medium is selected from the group consisting of a spray, a soak, an emollient salve, a lotion, a gel, a roll-on, and an imbued disposable.

3. The topical therapeutic product of claim 2, wherein the imbued disposable is selected from the group consisting of a panty liner, a wipe, an eye patch, a diaper, an adhesive bandage, and a trans-epi-dermal patch.

4. The topical therapeutic product of claim 2, wherein the spray is a nasal spray, a throat spray, or a body spray.

5. The topical therapeutic product of claim 1, wherein the topical therapeutic is an admix for a skincare product.

6. The topical therapeutic product of claim 5, wherein the skincare product is selected from the group consisting of a sunscreen, a moisturizer, a facial cream, a facial mask, a body lotion, a hand lotion, and a scalp moisturizer.

7. A method of alleviating pain and promoting cell and tissue repair from epidermal and mucosal injuries and disorders comprising:
a. administering the topical therapeutic product of claim 1 to an injury area;
b. delivering a pharmaceutically effective amount of the topical therapeutic to the surface of the injury area, thereby reducing pain from epidermal and mucosal injuries; and optionally
c. re-administering the topical therapeutic to the injury area.

8. The method of claim 7, wherein the injury area is selected from the group consisting of general epidermis, scalp, nasal cavity, mouth, throat, eyes, semen excretory site, urine excretory site, stool excretory site, vaginal site, and combinations thereof.

9. The method of claim 7, wherein the injury is selected from the group consisting of dermatitis, psoriasis, canker sores, cold sores, burns, inflammation, and combinations thereof.

10. The method of claim 9, wherein dermatitis is selected from the group consisting of atopic dermatitis, contact dermatitis, seborrheic dermatitis, and combinations thereof.

11. The method of claim 9, wherein the burn injury is selected from the group consisting of sunburn, thermal burn, radiation burn, chemical burn, electrical burn, and combinations thereof.

12. The method of claim 11, wherein the radiation burn further comprises injury caused by cancer radiation therapy.

13. The method of claim 11, wherein the chemical burn further comprises injury caused by excreted chemicals in body fluids from cancer therapy.

14. The method of claim 13, wherein the cancer therapy is selected from the group consisting of hormone therapy, chemotherapy, monoclonal antibody therapy, and combinations thereof.

15. The method of claim 13, wherein the body fluid is selected from the group consisting of sweats, tears, semen, vaginal discharge, urine, stool, and combinations thereof.

16. The method of claim 11, wherein a degree of the burn injury is selected from the group consisting of a first-degree burn or a second-degree burn.
